(19) United States
(12) Patent Application Publication
(10) Pub. No.: US 2005/0064015 A1
(43) Pub. Date: Mar. 24, 2005

(54) Methods and compositions for use in pet breeding

(76) Inventors: Stephanie Krammer, Lorrach-Hauingen (DE); Joachim Pheiffer, Lorrach (DE)

Correspondence Address: Stephen M Haracz;Bryan Cave, 1290 Avenue of the Americas, New York, NY, 10104, US

(21) Appl. No.: 10/497,714

(22) PCT Filed: Nov. 25, 2002
(86) PCT No.: PCT/EP02/13193

(30) Foreign Application Priority Data
Dec. 5, 2001 (EP) 01128899.0

(51) Int. Cl. A61K 38/40; A23K 1/165; A23K 1/17

(52) U.S. Cl. 424/442; 514/6

(57) ABSTRACT

Methods and compositions for use in pet breeding and growth including a method of improving the health status of newborn and young pets by the use of lactoferrin and/or lactoferricin and a feed composition for pets comprising lactoferrin and/or lactoferricin.

The present invention relates to methods and compositions for use in pet breeding and growth.

More particularly the present invention, in one aspect, is concerned with a method of improving the health status of newborn and young pets by the use of lactoferrin and/or lactoferricin. In another aspect the present invention is concerned with a feed composition for pets comprising lactoferrin and/or lactoferricin for improving the health status of newborn and young pets.

It will be understood that the term “pets” embraces all mammalian animals kept as pets such as carnivores, e.g. dogs and cats, rodents, e.g., chinchillas, guinea pigs, degus, mice, gerbils, hamsters, rats, ferrets, and lagomorphes, e.g. rabbits. For the purpose of the present invention, dogs and cats, particularly dogs, are of primary interest. The term “improving the health status” refers to the general condition of the animals, and comprises e.g. enhancing the immune system, stabilizing the gut flora or reducing the mortality, and, specifically, refers to the avoidance and suppression of diseases such as bacterial and viral infections, and inflammations as well as to development and sustaining of normal intestinal bacterial flora. The term “young” in context with pets, especially dogs, refers preferably to an age up to about 16 weeks.

Lactoferrin is a glycoprotein which is found in human breast milk and which has been identified also in the milk of a number of mammalian species. While there seem to exist different lactoferrins depending on the species of origin, the term lactoferrin as used herein denotes all lactoferrins having recognized essential characteristics of the lactoferrins, e.g. a single chain polypeptide of about 80000 D containing 1-4 glycan units and having iron-chelating properties. Lactoferrin formulations are commercially available, e.g., from Tatua Nutritionals: Tatua Co-operative Dairy Company Limited, State Highway 26 Tatuanui, Private Bag 800, Morrinsville, New Zealand. Lactoferricin is the product of peptic hydrolysis of lactoferrin. For the purposes of the present invention, lactoferrin is of primary interest.

In pet breeding, especially in breeding dogs and cats, the first weeks and months after birth are particularly critical to the survival of the young animals. For instance, in a study with dogs encompassing 2629 puppies, 42% died within the first 7 days after birth; 15% died between day 8 and 21, and 11% died between day 22-49. In accordance with the present invention it has been found that the administration to newborn or young puppies of lactoferrin and/or lactoferricin improves the health status of of the puppies and reduces their mortality. Thus, in accordance with the invention, lactoferrin and/or lactoferricin are administered to newborn or young pets, suitably during the first four months after birth, either directly or via their lactating mothers. Further, it has been found that administration of lactoferrin and/or lactoferricin to pregnant pets reduces the number of stillbirths.

In one aspect, the present invention is concerned with the use of lactoferrin and/or lactoferricin for improving the health status of newborn or young pets, particularly for reducing the mortality in newborn or young pets. In accordance with that aspect of the invention, lactoferrin and/or lactoferricin are administered to the pregnant or lactating pet and/or to the newborn or young pet preferably in dosages of from about 0.1 mg/kg body weight to about 200 mg/kg body weight per day. In a more preferred embodiment, lactoferrin and/or lactoferricin, especially lactoferrin, are administered in dosages of from about 0.1 mg/kg body weight to about 100 mg/kg body weight per day, particularly of from about 0.1 mg/kg body weight to about 40 mg/kg body weight per day.

Basically, small animals have a higher feed consumption per kg body weight per day than large animals. Further, the required energy or feed supply of the bitch increases by a factor of about 1.5 from week 5 to 9 of pregnancy, by a factor of about 2 in the first week after birth, by a factor of about 3 during week 2 to 5 after birth and by a factor of from abot 2.5 to about 1.25 during week 5 to 8 after birth. Therefore, the concentration of lactoferrin and/or lactoferricin in the feed and the amount of feed composition provided by the present invention will have to be adjusted accordingly to supply the appropriate amount of lactoferrin and/or lactoferricin to the animal.

The desired dosage of lactoferrin and/or lactoferricin can be administered by any conventional means, e.g., as a veterinary formulation for enteral or parenteral application or, preferably, as a feed supplement.

As set forth above, the present invention in one aspect is concerned with the use of lactoferrin and/or lactoferricin as a feed supplement for pregnant, lactating or newborn or young pets to improve the health status, particularly to reduce the mortality of the newborn or young pets. In accordance with that aspect of the invention, lactoferrin and/or lactoferricin are suitably used in an amount of from about 30 to about 1000 ppm (parts per million), more preferably of from about 30 to 960, especially from about 30 to 120 ppm based on the total feed composition.

In still another aspect, the invention is concerned with a pet feed composition, especially for pregnant, lactating or newborn or young dogs and cats comprising lactoferrin and/or lactoferricin for improving the health status and reducing the mortality of newborn or young pets.

The preferred pet feed composition in accordance with the present invention contains lactoferrin and/or lactoferricin in an amount of from about 30 to about 1000 ppm, more preferably of from about 30 to 960, especially from about 30 to 120 ppm based on the total feed composition.

In accordance with the invention, lactoferrin and/or lactoferricin may be administered to the unweaned pet, e.g. newborn or unweaned dog or cat. Alternatively, lactoferrin and/or lactoferricin may be administered to the pregnant or lactating animal, e.g. dog, thus providing a sufficient level of these compounds in the milk of the lactating animal for ingestion by the puppy. Further in accordance with the invention, lactoferrin and/or lactoferricin may be administered to the puppy after weaning to improve their health status and to reduce their mortality.

The feed composition according to the present invention may be any conventional pet feed and may be in a dry, canned, semi-moist or baked form. Preferably, the feed composition of the present invention is a dry feed composition. Typical components of such compositions are crude protein, crude fat, carbohydrates (NfE), starch, crude fibers, and ash, further on minerals, trace elements, vitamins, fatty acids, protein and amino acids, choline, carnitin, dietary fiber and substances required for balanced diets of the different animal species. Basic ingredients of such feed compositions are
Crude protein which comprises proteins and N-containing compounds of non-proteinaceous nature, e.g. acid amides, amines, free amino acids, ammonium salts, alkaloids;
Crude fat which comprises neutral fats, lipoids (phosphor sphingolipids, steroids and carotinoids) and other ethersoluble compounds;
N-free Extractions (NFE) which comprise polysaccharides (starch, glycogen), soluble saccharides (glucoce, fructose, saccharose, lactose, maltose and oligosaccharides) and soluble parts of cellulose, hemicellulose, lignin and pectine;
Crude fibers which comprise insoluble parts of cellulose, hemicellulose, lignin and other components of the cell wall like suberin, cutin etc.;
Ash which comprises minerals (macrominerals such as calcium, phosphorus, sodium, chloride, potassium, magnesium, and microminerals, i.e., trace elements, such as iron, copper, manganese, zinc, iodine, selenium, and further inorganic substances e.g. silicate;
Vitamins which comprise the vitamins A, B₁, B₂, B₆, B₁₂, C, D, E, K, as well as carotenoids like beta-carotene, lutein, zeaxanthin or lycopene, pantothenic acid, niacin, biotin, folic acid, linolic acid and choline.

Further components may, e.g. be omega-6-fatty acids, omega-3-fatty acids, L-carnitine, chondroitin sulfate, glucosamine, glutamine/glutamic acid, arginine, taurine and hydroxyproline.

Typical components which provide the ingredients for a dog feed composition may comprise, e.g., chicken/beef/turkey, liver, broken pearl barley, ground corn, crude fat, whole dried egg, fowl protein hydrolyzate, vegetable oil, calcium carbonate, choline chloride, potassium chloride, iodinized salt, iron oxide, zinc oxide, copper sulfate, manganese oxide, sodium selenite, calcium iodate, provitamin D, vitamin E, vitamin B₁, niacin, calcium panthothenate, pyridoxin hydrochloride, riboflavin, folic acid, biotin, vitamin B₁₂.

Dry pet feed may contain between 6 and 10% moisture and 90% or more dry matter.

A typical feed for adult dogs may, e.g. comprise at minimum 25% protein, 12% fat, 41.5% starch, 2.5% fibre, 1% linolic acid, 1% calcium, 0.8% phosphorus, the Ca:P ratio being 1:1, 0.6% potassium, 0.35% sodium, 0.09% chloride, 0.1% magnesium, 170 mg/kg of iron, 35 mg/kg of copper, 70 mg/kg of manganese, 220 mg/kg of zinc, 4 mg/kg of iodine, 0.43 mg/kg of selenium, 15000 IU/kg of vitamin A, 1200 IU/kg of vitamin D, 400 mg/kg of vitamin E, 11 mg/kg of vitamin B₁, 6 mg/kg of riboflavin, 30 mg/kg of pantothenic acid, 20 mg/kg of niacin, 4.3 mg/kg of pyridoxine, 0.9 mg/kg of folic acid, 0.2 μg/kg of vitamin B₁₂, 2500 mg/kg of choline, 0.8 mg/kg biotin, 2500 mg/kg cholin, all percentages being based on dry weight of the total feed composition.

Typical components which provide the ingredients for a dog feed composition for pregnant and lactating dogs and weaned puppies may comprise, e.g., chicken meat, rice, ground corn, crude fat, whole dried egg, fowl protein hydrolyzate, soy bean meal, broken soy bean, vegetable oil, fish oil, dicalcium phosphate, choline chloride, potassium chloride, iodinized salt, iron oxide, zinc oxide, copper sulfate, manganese oxide, sodium selenite, calcium iodate, vitamin A, provitamin D, vitamin E, niacin, thiamin, calcium panthothenate, pyridoxin hydrochloride, riboflavin, folic acid, biotin, vitamin B₁₂.

The feed composition for a pregnant, lactating or weaned dog may be adapted according to the specific requirements of the animal concerned, e.g. up to 1.8 MJ metabolizable energy per 100 g dry matter; the ratio of metabolizable raw protein (g) to MJ of metabolizable energy being about 10:1.

In order to achieve a sufficient energy density in the feed and to cover the requirements for essential fatty acids, at least 10% of fat maybe provided. A minimum of 10-20% of the given energy may emanate from carbohydrates because for the lactose synthesis larger amounts of glucose are needed. About 50% of the raw protein contained in the feed may be of animal origin to make sure that essential amino acids are supplied as required.

A typical feed for pregnant and lactating dogs and weaned puppies may, e.g. comprise at minimum 30% protein, 22% fat, 24% starch, 2.5% fibre, 1.15% calcium, 0.95% phosphorus, the Ca:P ratio being 1:1, 0.6% potassium, 0.35% sodium, 0.09% chloride, 0.1% magnesium, 260 mg/kg of iron, 30 mg/kg of copper, 55 mg/kg of manganese, 170 mg/kg of zinc, 4 mg/kg of iodine, 0.43 mg/kg of selenium, 15000 IU/kg of vitamin A, 1200 IU/kg of vitamin D, 120 mg/kg of vitamin E, 11 mg/kg of vitamin B₁, 6 mg/kg of riboflavin, 40 mg/kg of pantothenic acid, 27 mg/kg of niacin, 6.5 mg/kg of pyridoxine, 1.2 mg/kg of folic acid, 0.29 mg/kg of vitamin B₁₂, 0.8 mg/kg biotin, 3000 mg/kg cholin, all percentages being based on dry weight of the total feed composition.

Typical components which provide the ingredients for a cat feed composition may comprise beef, chicken meat, dried chicken liver, lamb meat, lamb liver, pork, turkey meat, turkey liver, poultry meal, fish meal, fowl protein hydrolysate, animal fats, plant oils, soy bean meal, pea bran, maize gluten, whole dry egg, ground corn, corn flour, rice, rice flour, dry sugar beet molasses, fructooligosaccharides, soluble fibres, plant gums, cellulose powder, clay, bakers yeast, iodized sodium chloride, calcium sulfate, sodium triphosphate, dicalcium phosphate, calcium carbonate, potassium chloride, choline chloride, magnesium oxide, zinc oxide, iron oxide, copper sulfate, iron sulfate, manganese oxide, calcium iodate, sodium selenite, provitamin D, vitamin E, thiamine, niacin, calcium pantothenate, pyridoxine hydrochloride, riboflavin, folic acid, biotin, vitamin B₁₂, taurin, L-carnitine, caseine, D-methionine.

A typical feed for adult cats may, e.g. comprise at minimum 32% protein, 15% fat, 27.5% starch, 11% dietetic fibres, 4.5% fibre, 3.4% linolic acid, 0.08% arachionic acid, 0.15% taurin, 50 mg/kg L-carnitin, omega 6/3=5, 1% calcium, 0.8% phosphorus, the Ca:P ratio being at least 1:1, 0.6% potassium, 0.4.% sodium, 0.6% chloride, 0.08% magnesium, 190 mg/kg of iron, 30 mg/kg of copper, 60 mg/kg of manganese, 205 mg/kg of zinc, 2.5 mg/kg of iodine, 0.2 mg/kg of selenium, 25000 IU/kg of vitamin A, 1500 IU/kg of vitamin D, 400 mg/kg of vitamin E, 30 mg/kg vitamin C, 20 mg/kg of vitamin B₁, 40 mg/kg of riboflavin, 56 mg/kg of pantothenic acid, 153 mg/kg of niacin, 14 mg/kg of pyridoxine, 3.2 mg/kg of folic acid, 0.2 mg/kg of vitamin B₁₂, 1 mg/kg of biotin, 3000 mg/kg of choline, all percentages being based on dry weight of the total feed composition.

Lactoferrin and/or lactoferricin may be incorporated into conventional pet feed e.g., into dry pet feed by spraying an aqueous solution of lactoferrin and/or lactoferricin on the feed composition while thoroughly mixing the composition.

The effect of the composition of the invention may be determined by various assays. The amount of bacteria may be determined which colonize the gut, e.g. E. coli, Enterococcus spec., Clostridia spec. and Lactobacillus spec. For example, samples of faeces or chymus may be taken and analyzed for the presence of microorganisms following procedures known to the skilled artisan. Samples obtainable by biopsy of the gut wall may be analyzed for changes, e.g., in gut wall structure or in the appearance of Peyer's plates.

The status of the immune system may be determined by analyzing blood samples for parameters like red blood cell count: Hemoglobin, hematocrit, number of erythrocytes, erythrocyte indices MCHC, MCH, MCV, and white blood cell count: Total number of leukocytes, differential blood count (Basophiles, neutrophiles, eosinophiles, monocytes, thrombocytes, B- and T-lymphocytes) relative and absolute. Changes in the humoral immune system may be followed by, e.g. determining IgA and IgG concentrations.

The antioxidative capacity in the whole organism is a means to reflect the organism's capability to withstand oxidative stress and protect cell membrane and cytosolic components against free radical damage. The higher the antioxidative capacity the higher its ability to neutralize free radicals and arrest the chain effect of free radical damage thereby playing a vital role in maintaining the health and integrity of individual cells.

Parameters for the determination of the antioxidant capacity are well known to the skilled artisan and include TEAC (Trolox equivalent antioxidant capacity) which may be measured, e.g. after a 1:1 dilution with a spectrophotometer, on which absorbance values may be recorded over 3 minutes, according to the method of Armstrong and Browne, Adv Exp Med Biol 366: 43-58 (1994), Total Antioxidant Capacity (TAC), Erythrocyte Superoxide Dismutase (SOD) which may, e.g., be determined based on a method coupling O₂-generators (xanthine and xanthine oxidase (XOD)) with an O₂-detector [2-(4-iodophenyl)-3-(4-nitrophenol)-5-phenyltetrazolium chloride] and monitoring absorbance in a spectrophotometer; Ferritin which may be determined by an enzyme-linked immunoassay, Ceruloplasmin which may be determined by a colorimetric method to determine ceruloplasmin oxidase activity; Vitamin E and C content; CK (creatine kinase); and AST (aspartate aminotransferase) which may be determined by using a fluorescence detector at a wavelength of 334 nm.

The following examples illustrate the invention further.

### EXAMPLE 1

Five portions of commercial dog feed (ROYAL CANIN Starter dog feed as supplied by Royal Canin Schweiz AG, CH-8105 Regensdorf, Switzerland) are sprayed with an aqueous solution of lactoferrin (as supplied by Tatua Nutritionals) in an amount sufficient to provide 30, 60, 120, 240 or 480 ppm (mg per kg), respectively, of lactoferrin in the final feed compositions. The feed compositions are thoroughly mixed to secure a uniform distribution of the feed supplement. The feed compositions have a dry matter content of about 90% by weight.

### EXAMPLE 2

Five portions of commercial puppy milk (ROYAL CANIN 1^{st} Age Milk) containing powdered milk (defatted, low in lactose), animal fat, plant oil, milk protein and lecithin, having an energy content of 5500 kcal ME per kg, are supplemented by adding with thoroughly mixing lactoferrin powder in an amount sufficient to provide 30, 60, 120, 240 or 480 ppm, respectively, of lactoferrin in the final milk powder compositions.

### EXAMPLE 3

Bitches (Beagle) weighing about 13 kg are grouped in three groups and each group is fed about 0.2 kg to about 0.34 kg per bitch per day of dog feed of Example 1 providing 30, 60 and 120 ppm, respectively, of lactoferrin from week 4 before giving birth, and are then fed about 0.4 kg to about 0.6 kg per day of dog feed of Example 1 providing 30, 60 and 120 ppm, respectively, of lactoferrin for 7 weeks after giving birth.

### EXAMPLE 4

Beagle puppies are grouped in three groups and each group is fed from their third day of life until week four the feed composition of Example 2 providing 30, 60 and 120 ppm, respectively, of lactoferrin (5-30 g of dry matter per kg body weight and day, the amount of feed increasing with the age of the animals). The animals are then weaned and fed 150-250 g (depending on age) per day of dog feed of Example 1 providing 30, 60 and 120 ppm) respectively, of lactoferrin for eight weeks after weaning.

### EXAMPLE 5

Beagle puppies, having a birth weight of about 240 g, are fed after week four about 180 g to about 250 g per day of dog feed of Example 1 providing 30, 60 and 120 ppm, respectively, of lactoferrin for eight weeks after weaning.

### EXAMPLE 6

Faeces of puppies of Example 4 receiving 60 ppm of lactoferrin and of puppies not receiving lactoferrin are collected after 4 and 12 weeks and analyzed for the presence of E. coli, Enterococcits spec., Clostridia spec. and Lactobacillus spec. The number of E. coli, Enterococcus spec. and Clostridia spec. is decreased and the number of Lactobacillits spec. is increased in the faeces of puppies receiving 60 ppm of lactoferrin.

### EXAMPLE 7

Chymus only is taken from puppies of Example 4 receiving 60 ppm of lactoferrin and of puppies not receiving lactoferrin at an age of 4 weeks. At an age of 12 weeks chymus and pieces of the gut wall are taken and stored in liquid nitrogen at −200° C. The samples are examined using light microscopy and immunohistochemistry for changes in gut wall structure and Peyer's plates. Lactoferrin has a beneficial effect on Peyer's plates and gut wall structure.

### EXAMPLE 8

Blood samples are taken from puppies of Example 4 receiving 60 ppm of lactoferrin and of puppies not receiving lactoferrin at an age of 4 and 12 weeks and effects on the immune system are analyzed by determining the number of red blood cells, leukocytes, B and T lymphocytes, the content of IgG, IgA and antibodies, the vitamin E and C content, and the TEAC. There is no effect on the number of red blood cells and leukocytes in puppies receiving 60 ppm of lactoferrin. The number of B and T lymphocytes, IgG, IgA and antibodies is increased as well as the TEAC, vitamin E and C content.

### EXAMPLE 9

Faeces of puppies of Example 5 receiving 60 ppm of lactoferrin and of puppies not receiving lactoferrin are collected after 8 and 12 weeks and analyzed for the presence of E. coli, Enterococcits spec., Clostridia spec. and Lactobacilltus spec. The number of E. coli, Enterococctus spec. and Clostridia spec. is decreased and the number of Lactobacilltis spec. is increased in the faeces of puppies receiving 60 ppm of lactoferrin.

### EXAMPLE 10

Chymus only is taken from puppies of Example 5 receiving 60 ppm of lactoferrin and of puppies not receiving lactoferrin at an age of 8 weeks. At an age of 12 weeks chymus and pieces of the gut wall are taken and stored in liquid nitrogen at −200° C. The samples are examined using light microscopy and immunohistochemistry for changes in gut wall structure and Peyer's plates. Lactoferrin has a beneficial effect on Peyer's plates and gut wall structure.

### EXAMPLE 11

Blood samples are taken from puppies of Example 5 receiving 60 ppm of lactoferrin and of puppies not receiving lactoferrin at an age of 8 and 12 weeks and effects on the immune system are analyzed by determining the number of red blood cells, leukocytes, B and T lymphocytes, the content of IgG, IgA and antibodies, the vitamin E and C content, and the TEAC. There is no effect on the number of red blood cells and leukocytes in puppies receiving 60 ppm of lactoferrin. The number of B and T lymphocytes, IgG, IgA and antibodies is increased as well as the TEAC, vitamin E and C content.

1-10. (Canceled).

11. A method for improving the health status of a newborn or young pet comprising administering lactoferrin and/or lactoferricin to a pregnant or lactating mother pet.

12. A method according to claim 11 wherein from about 0.1 mg/kg body weight to about 200 mg/kg body weight per day of lactoferrin and/or lactoferricin is administered.

13. A method of supplementing a feed composition for a pregnant and/or lactating pet comprising admixing lactoferrin and/or lactoferricin with a feed composition.

14. A method according to claim 13 wherein the amount of lactoferrin and/or lactoferricin in the feed composition is from about 30 to about 1000 ppm.

15. A pet feed composition for a pregnant and/or lactating mother pet in order to improve the health status in a newborn or young dog or cat comprising lactoferrin and/or lactoferricin.

16. A pet feed composition according to claim 15, wherein the amount of lactoferrin and/or lactoferricin is from about 30 to about 1000 ppm based upon the total composition.

17. A pet feed composition according to claim 16 comprising lactoferrin and/or lactoferricin in an amount of from about 30 to about 480 ppm.

18. A method for reducing the mortality of a newborn or young pet comprising administering lactoferrin and/or lactoferricin to a pet.

19. A method according to claim 18 wherein from about 0.1 mg/kg body weight to about 200 mg/kg body weight per day of lactoferrin and/or lactoferricin is administered to a pet selected from the group consisting of an unweaned pet, a weaned pet, and a pregnant or lactating mother pet.

20. A method according to claim 18 wherein the lactoferrin and/or lactoferricin is administered as a feed supplement in a feed composition.

21. A method according to claim 20 wherein the amount of lactoferrin and/or lactoferricin in the feed composition is from about 30 to about 1000 ppm.

22. A method according to claim 20 or 21 wherein the pet is a pregnant, lactating, newborn or young pet.

23. A pet feed composition for reducing the mortality of a newborn or young pet comprising lactoferrin and/or lactoferricin.

24. A pet feed composition according to claim 23 wherein the amount of lactoferrin and/or lactoferricin is from about 30 to about 1000 ppm based upon the total composition.

25. A pet feed composition according to claim 24 comprising lactoferrin and/or lactoferricin in an amount of from about 30 to about 480 ppm.

26. A pet feed composition according to any one of claims 23 to 25 wherein the pet is selected from the group consisting of an unweaned pet, a weaned pet and a pregnant or lactating mother pet.

27. A method of developing and sustaining normal intestinal bacterial flora in a newborn or young pet comprising administering lactoferrin and/or lactoferricin.

28. A method according to claim 27 wherein from about 0.1 mg/kg body weight to about 200 mg/kg body weight per day of lactoferrin and/or lactoferricin is administered to a pet selected from the group consisting of an unweaned pet, a weaned pet and a pregnant or lactating mother pet.

29. A method according to claim 27 wherein the lactoferrin and/or lactoferricin is administered as a feed supplement in a feed composition.

30. A method according to claim 29 wherein the amount of lactoferrin and/or lactoferricin in the feed composition is from about 30 to about 1000 ppm.

31. A method according to claim 29 or 30 wherein the pet is a pregnant, lactating, newborn or young pet.

32. A pet feed composition for developing and sustaining normal intestinal bacterial flora in a newborn or young pet comprising lactoferrin and/or lactoferricin.

33. A pet feed composition according to claim 32 wherein the amount of lactoferrin and/or lactoferricin is from about 30 to about 1000 ppm based upon the total composition.

34. A pet feed composition according to claim 33 comprising lactoferrin and/or lactoferricin in an amount of from about 30 to about 480 ppm.

35. A pet feed composition according to any one of claims 32 to 34 wherein the pet is selected from the group consisting of an unweaned pet, a weaned pet and a pregnant or lactating mother pet.
